(19) United States
(12) Patent Application Publication
(10) Pub. No.: US 2023/0016023 A1
(43) Pub. Date: Jan. 19, 2023

(54) PROSTHESIS SOCKET AND METHOD FOR PRODUCING SAME

(71) Applicant: OTTOBOCK SE & CO, KGAA, Duderstadt (DE)

(72) Inventors: Andreas LEINIGER, Birkungen (DE); Jens VOLKMAR, Gerblingerode (DE); Uli MAIER, Berlin (DE); Coerte VOORHEES, Berlin (DE)

(73) Assignee: OTTOBOCK SE & CO, KGAA, Duderstadt (DE)

(21) Appl. No.: 17/786,419

(22) PCT Filed: Dec. 15, 2020
(86) PCT No.: PCT/EP2020/086220

§ 371 Date: Jun. 16, 2022

(30) Foreign Application Priority Data
Dec. 18, 2019 (DE) 10 2019 134 986.9

(51) Int. Cl. A61F 2/80; B33Y 80/00

(52) U.S. Cl. CPC A61F 2/80; B33Y 80/00; A61F 2002/5049

(57) ABSTRACT

A prosthesis socket having an open proximal end for accommodating an amputation stump, an inner wall and an outer wall, at least one hollow space being located between the inner wall and the outer wall and at least one internal element extending through said hollow space, the at least one internal element being integrally formed with the inner wall and/or the outer wall.

### CROSS REFERENCE TO RELATED APPLICATIONS

This is a national phase application of International Application No. PCT/EP2020/086220, filed 15 Dec. 2020, which claims the benefit of German Patent Application No. 10 2019 134 986.9, filed 18 Dec. 2019, the disclosures of which are incorporated herein, in their entireties, by this reference.

### TECHNICAL FIELD

The invention relates to a prosthesis socket with an open proximal end for accommodating an amputation stump, an inner wall and an outer wall.

### BACKGROUND

This type of prosthesis socket has been known from the prior art for many years and is used for various prostheses and amputation stumps. They act as a connecting element between the amputation stump of the patient and the prosthesis, which replaces a body part of the patient that has been amputated. In the case of leg prostheses in particular, prosthesis sockets are subjected to a significant mechanical load. This is true for both upper leg prostheses and lower leg prostheses.

To ensure that it is both comfortable to wear and suitably safe for the patient, the socket must be securely arranged on the amputation stump. A range of possibilities for achieving this are known from the prior art. A so-called liner is often pulled over the amputation stump before the combination of amputation stump and liner is inserted into the prosthesis socket. The liner can interact with a recess provided for this purpose in the prosthesis socket via a fastening pin, which is usually located at the distal end of the liner. As an alternative, a negative pressure is created between the liner and the socket in a confined volume, securing the socket to the liner and thus to the amputation stump.

The wearer of the prosthesis socket that uses the prosthesis to walk must raise the entire prosthesis, including socket, during the swing phase of each step and swing it forwards. The prosthesis socket must not make the wearer feel that the connection between socket and amputation stump is not secure. At the same time, the prosthesis socket must feature a connection element for further prosthesis elements at its distal end, for example a prosthetic knee or a lower leg with an ankle and foot arranged thereon. This connection, created via the connection element, must safely transfer the forces that occur during walking and make the wearer feel that it is secure.

The various elements of a prosthesis socket are therefore designed to be relatively solid so they can withstand the loads that occur. As a result, the prosthesis socket in particular is heavy, rendering it less comfortable to wear.

Modern prosthesis sockets are often produced using carbon fiber composite materials. Such sockets are light yet still offer significant mechanical stability, making them very well-suited to meet the demands of a prosthesis socket. In recent years, prosthesis sockets have also been produced using an additive manufacturing process, for example by means of a 3D printer. This enables new designs and shape of prosthesis socket that could not be produced using conventional production methods, such as carbon fiber composite materials.

WO 2017/012888 A1 discloses a production method for a prosthesis socket in which an inner socket is first produced using a 3D printer. However, the material used is unable to absorb and withstand the mechanical loads of a prosthesis socket for treating a lower limb. To resolve this issue, additional reinforcement elements are applied to points that are particularly subjected to loads on the outside of the inner socket produced in this way. However, the production method becomes more complex as a result, as it requires multiple steps and several different materials.

### SUMMARY

The invention therefore aims to prevent or at least reduce the disadvantages of the prior art, and to propose a prosthesis socket and method for its production that is light and able to withstand the loads.

The invention solves the problem addressed by way of a prosthesis socket according to the preamble of claim 1, characterized in that at least one cavity is arranged between the inner wall and the outer wall, through which at least one internal element extends, the at least one internal element being designed as a single piece with the inner wall and/or outer wall.

The design of the prosthesis socket according to the invention with a cavity reduces the weight, so that the prosthesis socket and thus the entire prosthesis is lighter. At the same time, the internal element that extends through the cavity increases stability. The internal element is designed as a single piece with the inner wall and is therefore produced in the same step of the method as the respective wall. Preferably, the inner wall, the outer wall and the at least one internal element are designed as a single piece.

The cavity is situated between the inner wall and outer wall, and is not the space in which the amputation stump can be accommodated. The cavity is preferably closed, so that there is no connection between the interior of the cavity and the surrounding area. The inner wall of the prosthesis socket is preferably at least roughly designed according to the shape of the amputation stump to be accommodated. It is essentially cup-shaped and the prosthesis socket preferably has a closed distal end. The cavity is preferably located distally to the distal end of the inner wall. The at least one cavity is preferably restricted in the proximal direction by the inner wall of the prosthesis socket and in all other directions by the outer wall of the prosthesis socket. It can be designed to be rotationally symmetric about a longitudinal axis; however, this is not essential. At the distal end of the prosthesis socket there is preferably a connection means for a further distal prosthesis component. The part of the prosthesis socket on which this connection means is located also forms part of the prosthesis socket and preferably restricts the at least one cavity in the distal direction. In terms of the cavity, the inner wall and outer wall have an inner side that faces towards the cavity. The at least one internal element is arranged in this inner side of the respective wall.

The at least one internal element extends through the at least one cavity. It not only protrudes into the at least one cavity, but passes through it, preferably completely. It preferably connects two walls that surround and restrict the at least one cavity. It can extend between the inner wall and the outer wall or connect multiple positions or points on a single wall, i.e. the inner wall or outer wall, to each other.

The at least one internal element is preferably a support element, which preferably extends from the inner wall to the outer wall. Such a support element is preferably a strut, a rod or a rod-like element, meaning that it has a greater extension in the longitudinal direction, i.e. from the inner wall to the outer wall, than in the other spatial directions. The cross-section may be circular, oval, square, triangular, polygonal or irregular. A support element may also be arranged between two points or positions on a single wall, i.e. the inner wall or the outer wall, and connect these points or positions with each other. The cross-section may vary along the course of the support element, in particular the diameter can change so that it is larger, for example, in the area of the connection between the wall and the support element. Of course, the cross-section may also be constant over the course of the support element.

It is especially preferable if the at least one support element at least also extends in the proximal-distal direction. The longitudinal direction of the at least one support element thus also has at least one component in this direction, this component preferably being the largest component. In this case, the at least one support element extends further in the proximal-distal direction than in all other directions.

Preferably, at least 25, preferably at least 100, especially preferably at least 500 support elements extend in the at least one cavity, wherein several, but preferably all, of them extend from the inner wall to the outer wall. This enables an even distribution of load on the various support elements and the stability of the prosthesis socket can be further optimized.

In a preferred embodiment, at least one support element is perpendicular to the inner wall and/or outer wall. Particularly preferably, several, most preferably all, support elements are perpendicular to the inner wall and/or the outer wall. This avoids shear moments and tilting moments on the individual support elements, which could otherwise occur when the prosthesis socket is subjected to a load, for example when walking. This also increases the stability of the prosthesis socket. As a result, the number and thickness of the support elements can be reduced, thereby saving on material and weight.

Preferably at least one internal element is a partition element that divides the cavity into two partial cavities. Such a partition element is preferably arranged exclusively on the outer wall of the prosthesis socket. In contrast to a support element, a partition element is preferably flat, i.e. it has a greater extension in two directions perpendicular to each other than in the third spatial direction, which is perpendicular to the first two directions. The at least one partition element preferably extends in one plane, which is essentially perpendicular to the proximal-distal direction. In this case, the at least one partition element is preferably arranged exclusively on the inner side of the outer wall.

The at least one partition element preferably features at least one opening, which connects the two partial cavities to each other. Particularly preferably, at least one support element extends through this opening.

In an alternative embodiment of a partition element, the partition element is designed to be three-dimensional, i.e. it is not exclusively flat in design. It may be designed as a hollow tube, curved surface or otherwise. By using a partition element with a closed cross-section contour, such as a tube, the at least one cavity is divided into two partial cavities, one of which is found inside the partition element and one outside. The cross-section contour may be circular, oval, square, triangular, polygonal or irregular. It is also possible for the contour to change in shape, size and/or contour along a longitudinal direction of the partition element formed in this way. A partition element designed in this shape preferably extends essentially along the proximal-distal direction, so that the partial cavities created by the partition element also extend in this direction. The partition element is preferably arranged between the inner wall in the proximal area of the cavity and the outer wall in the distal area of the cavity.

In a preferred embodiment, the prosthesis socket has a plurality of partition elements, so that the at least one cavity is divided into a plurality of partial cavities. The partition elements preferably run parallel to one another and, particularly preferably, these partial elements are designed to be flat and, more preferably, run perpendicular to the proximal-distal direction. This results in the creation of chambers that considerably increase a bending stiffness of the prosthesis socket in this area.

Preferably, at least one connection means for a distal prosthesis component is arranged at a distal end of the prosthesis socket. The connection means is preferably arranged on the outer wall of the prosthesis socket. The distal prosthesis component is, for example, a prosthetic knee, a lower leg tube or a prosthetic foot, preferably with an artificial ankle joint. In particular, but not only in the case of lower leg prosthesis sockets, it is beneficial to enlarge an expansion of the prosthesis socket in the distal direction to such an extent, i.e. to extend the prosthesis socket in this direction to such an extent, that a prosthetic foot and/or artificial ankle joint can be positioned directly on the connection means. This reduces the number of prosthesis components required and simplifies assembly of the prosthesis. For example, a separate lower leg tube is no longer needed. The at least one cavity is at least also located in the area of the prosthesis socket extended in this manner, said cavity preferably being divided into multiple partial cavities or chambers by at least one, but preferably multiple, partition elements.

If the prosthesis socket features multiple support elements, imaginary extensions of the support elements run through the connection means. Particularly preferably, they meet at a single point within the connection means. In this case, the force transmission through the support elements takes place directly into the connection means and thus directly into a prosthetic component arranged on the connection means. This also renders it possible to prevent or at least reduce shear moments or tilting moments.

The prosthesis socket is preferably a prosthesis socket for a prosthesis for treating a lower limb, preferably for a lower leg prosthesis.

A prosthesis socket of the type described here preferably has at least a first insert element, which has a contact surface that is designed to at least partially, but preferably completely, correspond to at least one part of the inner wall of the prosthesis socket. Such an insert element is also beneficial in other prosthesis sockets that do not have a cavity and/or internal element, for example. Therefore, such an insert element in combination with the features of the preamble of the original claim 1 constitutes a separate invention that can be used with or without the features of the prosthesis sockets described here. An insert element is used when the amputation stump loses volume over time and the original prosthesis socket no longer optimally fits the amputation stump. Then an insert element is produced, which must be laboriously adapted, for example ground, to the desired shape. This time-consuming and therefore cost-intensive.

With the present invention, this becomes easier. The prosthesis socket is preferably produced in an additive manufacturing process, such as a 3D printing process, so that its contours and shaped are stored electronically. Therefore, a first insert element can easily be produced, in particular also 3D printed, the contact surface of which is optimally adapted to the inner wall of the prosthesis socket, preferably its outer side opposite the inner side. The insert element can preferably be placed with its entire surface against the outer side of the inner wall of the prosthesis socket. The insert element is thus introduced into the cavity, which is provided for accommodating the amputation stump. Complex post-treatment is not necessary. In the desired position, the insert element is glued or otherwise fixed to the inner wall of the prosthesis socket. Preferably, the insert element then forms a uniform and, in particular, continuous, i.e. stepless, surface with the part of the outer wall of the inner wall of the prosthesis socket that is not covered by it.

The prosthesis socket preferably has at least a second insert element that features a contact surface, which is designed to correspond to the inner wall of the prosthesis socket and/or a surface of the first insert element that is opposite the contact surface. The prosthesis socket is preferably delivered with several such insert elements, preferably forming a set with said elements. Alternatively or additionally, insert elements with the appropriate material thickness may be produced individually. If a permanent change in volume of the amputation stump occurs, particularly a reduction in volume, this can be compensated by the careful selection of one or multiple insert elements.

The invention also solves the problem addressed by way of a method for producing a prosthesis socket as described here, wherein with said method:
the directions of the forces expected to occur during use of the prosthesis socket are determined,
on the basis of these directions, the positions and directions of the at least one support element are determined, and
on the basis of these positions and directions of the support elements the prosthesis socket is produced.

To be able to determine the directions of the forces expected to occur during use, a model of the prosthesis to be produced is preferably generated, wherein said model can be edited in an electronic data processing device. It is preferably stored in an electronic data storage device. With the aid of a saved model, a simulation unit of the electronic data processing device then simulates the forces that occur and stores their values and directions. On the basis of this data, a modelling unit of the electronic data processing device models the optimal positions and orientations of the support elements required. In addition, the thickness, cross-section, course and/or number of support elements can be used as an optimization parameter of the modelling unit. The electronic data processing device then transmits control commands to the additive manufacturing installation, for example the 3D printer, and controls it in such a way that the modelled prosthesis socket is printed.

Preferably, the prosthesis socket is at least partially produced by means of an additive manufacturing process, for example using a 3D printer, the at least one support element in particular being produced using additive manufacturing, for example printed.

### BRIEF DESCRIPTION OF THE DRAWINGS

In the following, some examples of embodiments of the present invention will be explained in more detail by way of the attached figures:

They show

FIG. 1—the schematic representation of a lower leg prosthesis,

FIG. 2—the representation from FIG. 1 with the prosthesis socket in a sectional view,

FIGS. 3-6—schematic sectional representations through prosthesis sockets,

FIGS. 7-8—schematic representations of insert elements,

FIG. 9—schematic sectional representations through the cavity,

FIG. 10—schematic representations of a connection means,

FIG. 11—the schematic sectional view through a socket with connection means,

FIG. 12—the schematic representation of a part of a prosthesis socket,

FIG. 13—the schematic sectional representation through a part of a prosthesis socket with connection means, and

FIG. 14—a schematic sectional representation through a prosthesis socket along another plane.

### DETAILED DESCRIPTION

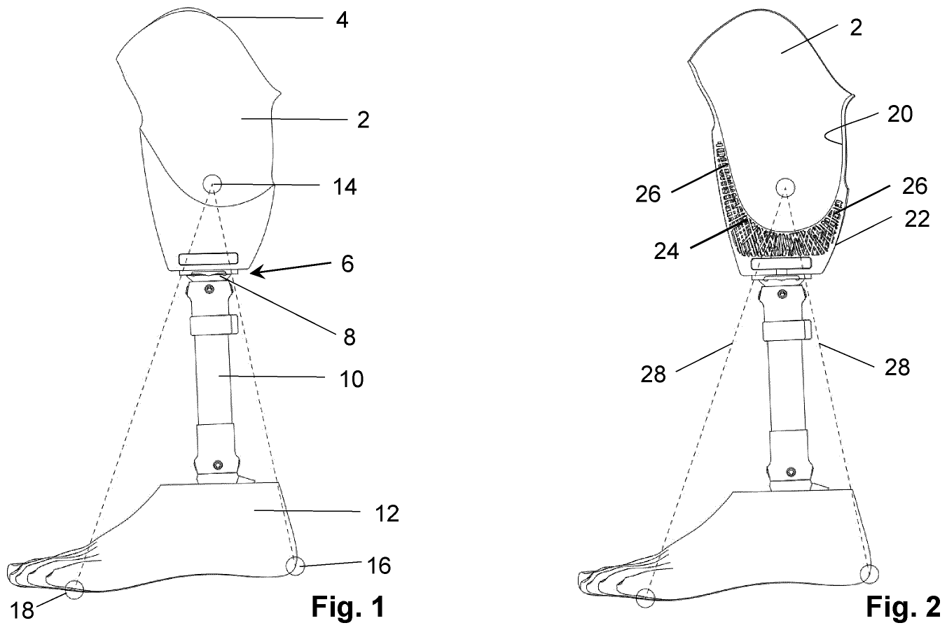

FIG. 1 shows the schematic representation of a lower leg prosthesis that comprises a prosthesis socket 2, which has an open proximal end 4 and a closed distal end 6. At the distal end 6 is a connection means 8, on which a lower leg tube 10 is arranged. A prosthetic foot 12 is located at the lower end of this lower end tube 10. To determine position and orientation, a virtual pivot axis 14 for a knee accommodated in the prosthesis socket 2 was assumed in the example of an embodiment shown. This pivot axis 14 was connected on the one hand to a heel strike point 16 and on the other to a point 18 at which the load is applied during the transition to the swing phase. This results in the maximum load angle.

FIG. 2 depicts the representation from FIG. 1, the prosthesis socket 2 now being shown in a sectional view. It has an inner wall 20 and an outer wall 22, between which there is a cavity 24. In the example of an embodiment shown, a plurality of support elements 26 is arranged in this cavity 4, of which run through the cavity 24. Most of them are arranged with one end on an inner side of the inner wall 20 and with the opposite end on the inner side of the outer wall 22. However, a small number extends between two different points of the inner side of the outer wall 22. The dashed lines 28 demonstrate that some of the support elements 26 extend along these lines 28, thereby enabling an optimal force transmission upon heel strike and when lifting the toes. The remaining support elements 26 have been configured in a similar manner. Different support elements are subjected to a maximum load for different loads during different movements and/or in different phases of a gait cycle. Said support elements extend in the optimal direction for force transmission at the respective moment.

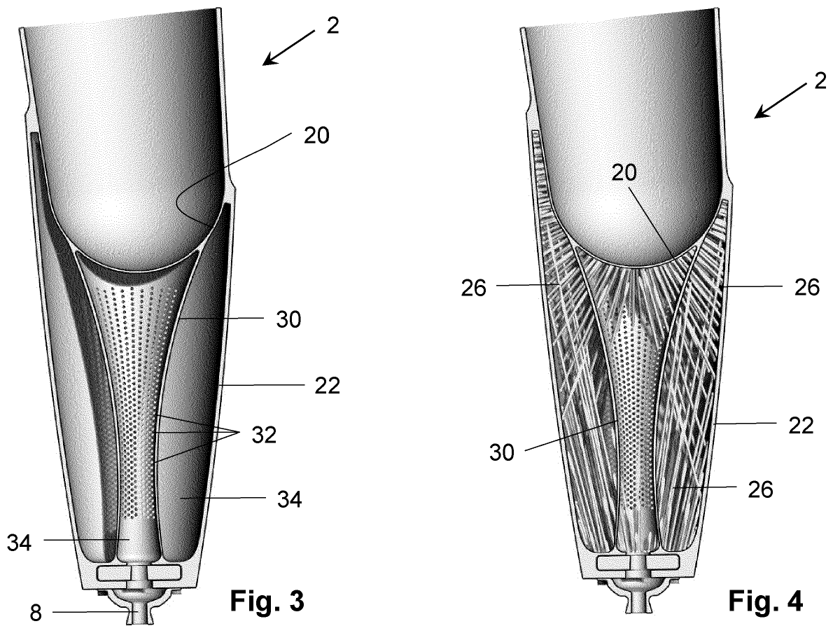

FIG. 3 shows a schematic sectional view through another prosthesis socket 2. It also has an inner wall 20 and an outer wall 22, between which there is a cavity 24. A partition element 30 is situated in the cavity, said partition element featuring a closed cross-section contour in the example of an embodiment shown. The partition element 30 has a plurality of openings 32, which are depicted as small dots. The partition element 30 extends from the inner wall 20 in the proximal area of the cavity 24 to the distal end of the cavity 24, where it ends at the outer wall 22. This creates two partial cavities 34, one of which is found outside of the partition element 30 between the partition element 30 and the outer wall 22. The other partial cavity 24 is located inside of the partition element 30. In FIG. 3, it can be recognized that the prosthesis socket 2 has been extended in the distal direction, so that the connection means 8, designed as a pyramid adapter in the example of an embodiment shown, is displaced further in the distal direction. If the prosthesis socket 2 schematically depicted in FIG. 3 is used for a lower leg prosthesis, as is shown in FIGS. 1 and 2, it is no longer necessary to use a separate lower leg tube 10. The extended distal area of the prosthesis socket 2 assumes these tasks and, due to the structures inside the cavity 24, is able to withstand the occurring loads.

FIG. 4 shows another representation of a prosthesis socket 2 with the inner wall 20, the outer wall 22 and the partition element 30 previously depicted in FIG. 3. In addition, the prosthesis socket 2 in FIG. 4 features a number of support elements 26. Of these support elements 26, some extend from the inner wall 20 to the outer wall 22, wherein some of these support elements 26 are guided through the openings 32 in the partition element 30. Some of the support elements 26 extend completely in the outer partial cavity 34 and extend from a first point of the outer wall 22 to a second point of the outer wall 22. Most of these support elements 26 extend at least primarily in the proximal-distal direction.

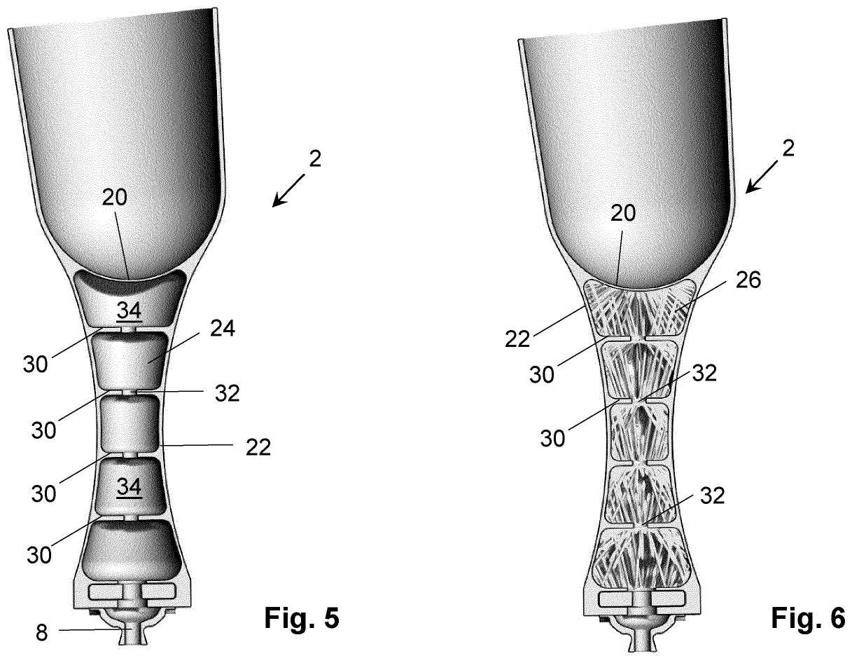

FIG. 5 depicts a further schematic representation of a prosthesis socket 2 in a sectional view. This prosthesis socket 2 also features the inner wall 20, the outer wall 22 as well as the connection means 8 for a distal prosthesis element. With the prosthesis socket 2 shown in FIG. 5, the distal area is also extended, thereby rendering, for example, a lower leg tube redundant. The cavity 24 is located between the inner wall 20 and the outer wall 22, said cavity being divided by four partition elements 30 into five partial cavities 34 in the example of an embodiment shown. The partition elements 30 run parallel to one another and are designed to be flat. Unlike the partition element 30 from FIGS. 3 and 4, it does not have a closed cross-section contour. The partition elements 30 in FIG. 5 each feature an opening 32, through which the respective adjacent partial cavities 34 are connected to each other.

In another sectional representation, FIG. 6 depicts a prosthesis socket 2, which is similar in structure to the prosthesis socket 2 shown in FIG. 5. It also features four partition elements 30, which divide the distal area of the prosthesis socket into partial cavities 34. In addition, the prosthesis socket 2 shown in FIG. 6 has a plurality of support elements 26, which partially extend through the openings 32 into the partition element 30. Others extend between two partition elements 30 or run from the inner wall 20 to the outer wall 22.

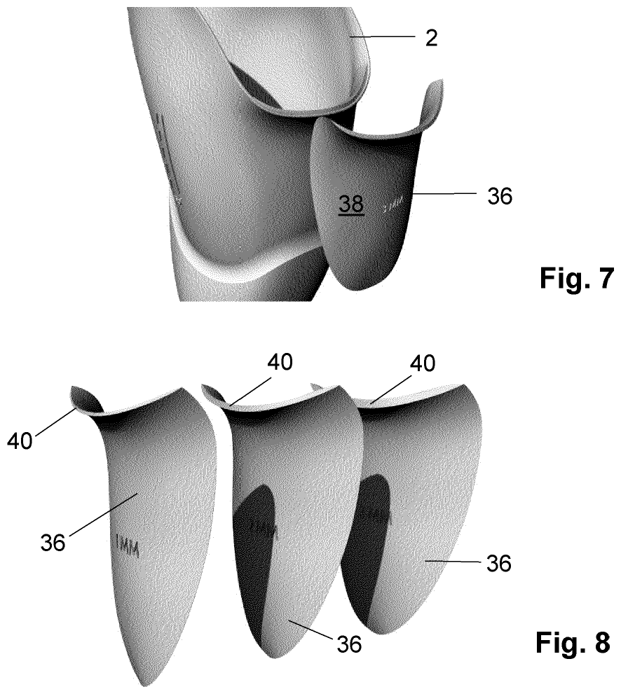

FIG. 7 schematically depicts a prosthesis socket 2 with an insert element 36 produced for this prosthesis socket 2. It features a contact surface 38, which is designed to correspond to an outer side of the inner wall 20 of the prosthesis socket 2, so that the insert element 26 can be arranged with the contact surface over the entire surface of the inner wall 20 of the prosthesis socket 2. This takes into account a reduction in volume of the amputation stump that is to be accommodated in the prosthesis socket 2.

FIG. 8 shows three different insert elements 36 of different thicknesses. The rim 40 shows this particularly clearly. Depending on the degree of volume reduction of the amputation stump, the appropriate insert element 36 can be used. The insert elements 36 are preferably 3D-printed, rendering them especially easy to adapt to the inner wall 20 of the prosthesis socket and to design correspondingly.

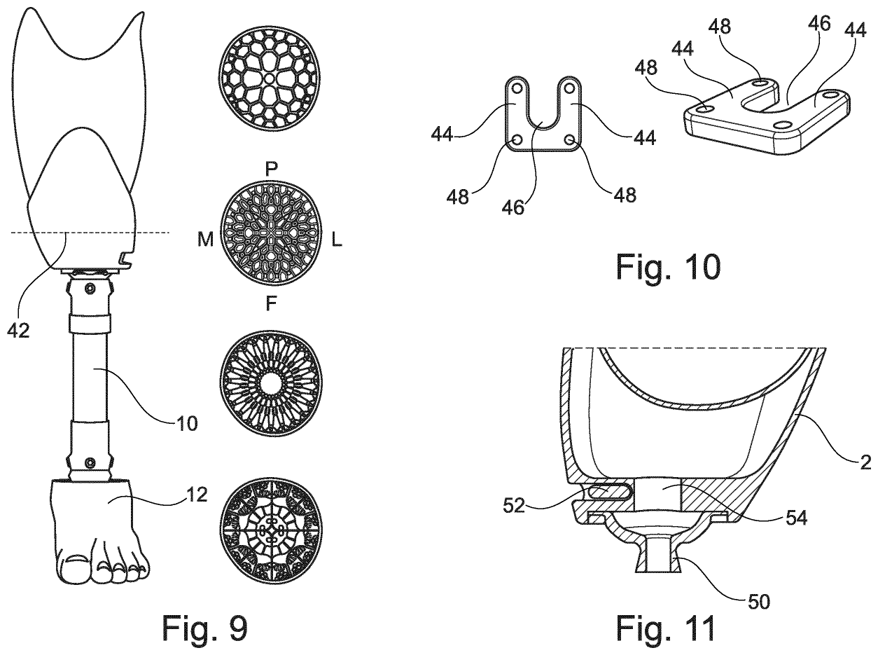

The left-hand part of FIG. 9 schematically depicts a frontal view of a lower leg prosthesis with the prosthesis socket 2, the lower leg tube 10 and the prosthetic foot 12. The right-hand part of FIG. 9 depicts four sectional representations through the prosthesis socket 2 along the dashed line 42. One can see partly complex patterns of support elements 26 and partition element 30, although these cannot be distinguished in the representations shown, as it is not clear between which two points the internal elements extend. In the four sectional views shown one above the other, the medial side is on the left, the lateral side is on the right, and the frontal side is at the bottom.

FIG. 10 schematically depicts the representation of a connection means 8 in two different perspectives. It features an essentially u-shaped design with two sides 44 and a recess 46 between them. In the example of an embodiment shown, the connection means 8 has four boreholes 48, which are preferably provided with an inner thread. In this way, screws can be fastened in them so that another connection means, for example a pyramid adapter 50, can be attached.

This is shown in FIG. 11. In the distal area of the prosthesis socket 2 shown there is a slit-shaped depression 52, which is preferably designed as a single piece with the rest of the prosthesis socket 2. It is thus preferably not inserted after the production of the prosthesis socket 2, for example by means of a milling machine, but rather is preferably designed as a single piece with the rest of the prosthesis socket 2 in an additive manufacturing process. The connection means 8 shown in FIG. 10 can be introduced into this depression, so that the pyramid adapter 50 shown can be screwed on. Here, a stern 54 transmitting the large loads is arranged in the recess 46 in the connection means 8, so that the at times large mechanical forces which can occur in particular with leg prostheses are transmitted.

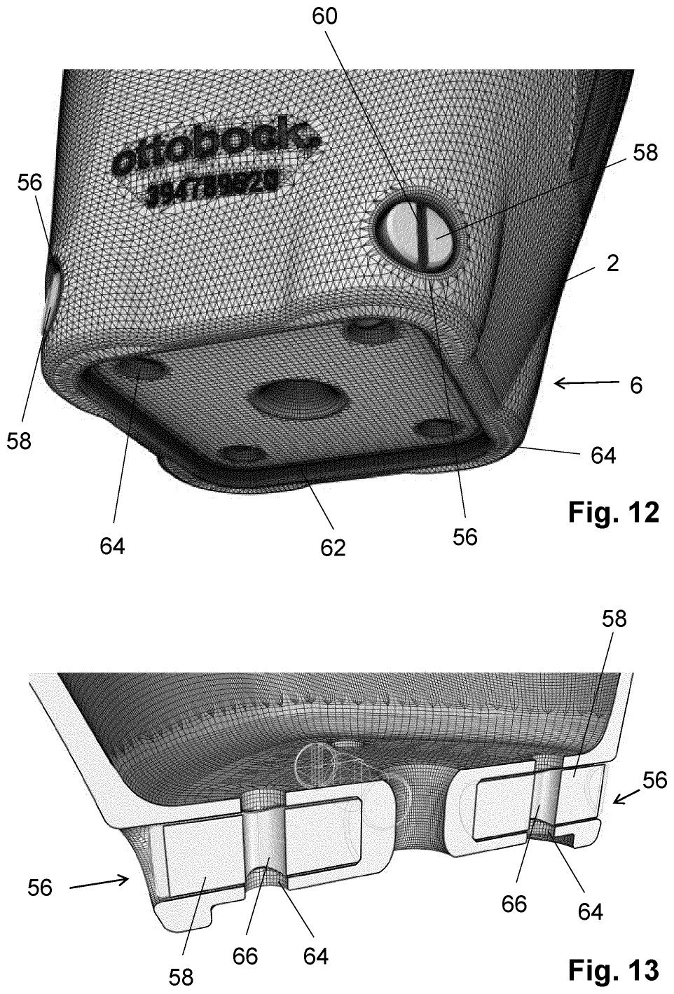

FIG. 12 shows a section of a prosthesis socket 2 and in particular its distal end 6. It features four recesses 56 that are arranged in the corner areas and of which two can be recognized in FIG. 12. The connection means 8 are contained within in the form of bolts 58. In the example of an embodiment shown, the bolts 58 feature a slit 60 on the side facing outwards, so that they can be moved by means of a suitable tool, such as a screwdriver. Four holes 64 are situated on a distal side 62 of the prosthesis socket 2, into each of which a screw can be inserted for attaching, for example, a pyramid adapter 50 not shown in FIG. 12. Particularly preferably, the bolts 58 feature a corresponding thread into which the screws can be screwed when the bolt 58 is arranged in the correct position and orientation in the recess 56.

FIG. 13 shows the situation in a sectional view. Two bolts 58 can be seen, each of which has been inserted into a recess 56. They each feature a bore 66, preferably designed with the thread, which is not depicted in FIG. 13. The screws can be inserted through the holes 64 in the distal end 62 of the prosthesis socket 2, wherein said screws preferably engage with the thread provided on the inner wall of the bore 66 and thus attach an adapter, such as a pyramid adapter 50, not depicted in FIG. 13.

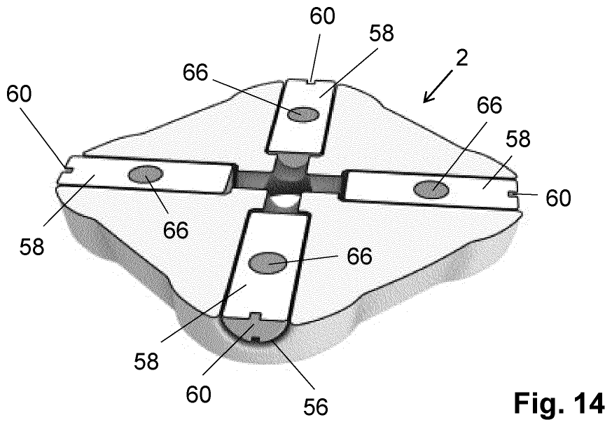

FIG. 14 depicts a further sectional representation through the prosthesis socket 2 parallel to the distal side 62 of the prosthesis socket 2. The four bolts 58 are each inserted into one of the recesses 56 and, as described above, feature the slit 60, so that their alignment and orientation relative to the rest of the prosthesis socket 2 and particularly relative to the holes 64 on the distal side 62 of the prosthesis socket 2 can be adjusted. Preferably, the desired position is very easy to achieve, for example by arranging the slit 60 perpendicularly, as depicted in FIG. 14. Each bolt 58 features the bores 66, which can be brought into overlap with the holes 64, not depicted in FIG. 14, on the distal side 62 of the prosthesis socket 2 by rotating the bolts 58 about their longitudinal axis, so that the screws, not depicted, for attaching an adapter, especially a pyramid adapter 50, can be screwed in.

### REFERENCE LIST

2 prosthesis socket

4 proximal end

6 distal end

8 connection means

10 lower leg tube

12 prosthetic foot

14 virtual pivot axis

16 heel strike point

18 point

20 inner wall

22 outer wall

24 cavity

26 support element

28 dashed line

30 partition element

32 opening

34 partial cavity

36 insert element

38 contact surface

40 rim

42 dashed line

44 side

46 recess

48 borehole

50 pyramid adapter

52 depression

54 stem

56 recess

58 bolt

60 slit

62 distal side

64 hole

66 bore

1. A prosthesis socket with
an open proximal end for accommodating an amputation stump,
an inner wall and
an outer wall,
wherein at least one cavity is arranged between the inner wall and the outer wall, through which at least one internal element extends, the at least one internal element being designed as a single piece with the inner wall and/or outer wall.

2. The prosthesis socket according to claim 1, wherein the at least one internal element is a support element, which preferably extends from the inner wall to the outer wall.

3. The prosthesis socket according to claim 2, wherein at least 25, preferably at least 100, especially preferably at least 500 support elements extend in the at least one cavity from the inner wall to the outer wall.

4. The prosthesis socket according to claim 2, wherein at least one support element, preferably a plurality of support elements, is perpendicular to the inner wall and/or perpendicular to the outer wall.

5. The prosthesis socket according to claim 1 wherein the at least one internal element is a partition element that divides the cavity into two partial cavities and is preferably arranged only on the outer wall.

6. The prosthesis socket according to claim 5, wherein the at least one partition element has at least one opening that connects the two partial cavities.

7. The prosthesis socket according to claim 6, wherein at least one support element extends through the at least one opening of the at least one partition element.

8. The prosthesis socket according to claim 5, wherein a plurality of internal elements are partition elements (30) that preferably run parallel to each other.

9. The prosthesis socket according to claim 1, characterized in that a connection means for a distal prosthesis component is arranged on a distal end of the prosthesis socket and imaginary extensions of a plurality of support elements in the connection means preferably meet at one point within the connection means.

10. The prosthesis socket according to claim 1, wherein the prosthesis socket is a prosthesis socket for a prosthesis for treating a lower limb, preferably a lower leg prosthesis.

11. The prosthesis socket according to claim 1, wherein the prosthesis socket comprises at least a first insert element that has a contact surface which is designed to correspond to the inner wall of the prosthesis socket.

12. The prosthesis socket according to claim 11, wherein the prosthesis socket has at least a second insert element that features a contact surface, which is designed to correspond to the inner wall of the prosthesis socket and/or a surface of the first insert element that is opposite the contact surface.

13. A method for producing a prosthesis socket according to claim 2, wherein:
the directions of the forces expected to occur during use of the prosthesis socket are determined,
on the basis of these directions, the positions and directions of the at least one support element (26) are determined, and
on the basis of these positions and directions of the support elements (26) the prosthesis socket (2) is produced.

14. The method according to claim 13, wherein the prosthesis socket is at least partially produced by means of an additive manufacturing process, wherein the at least one support element in particular is produced in the additive manufacturing process.

15. A prosthesis socket for a lower leg prosthesis comprising:
an open proximal end to accommodate an amputation stump, an inner wall, and an outer wall;
at least one cavity arranged between the inner wall and the outer wall;
a plurality of internal elements;
wherein the plurality of internal elements are support elements which extend through the at least one cavity, each internal element being designed as a single piece with the inner wall and/or outer wall; and
wherein a plurality of the support elements extend through the at least one cavity from the inner wall to the outer wall.

16. The prosthesis socket of claim 15, wherein at least 25, at least 100, or at least 500 support elements extend through the at least one cavity from the inner wall to the outer wall.

17. The prosthesis socket of claim 15, wherein a plurality of support elements is perpendicular to the inner wall and/or perpendicular to the outer wall.

18. A prosthesis socket for a lower leg prosthesis comprising:
an open proximal end to accommodate an amputation stump, an inner wall, and an outer wall;
at least one cavity arranged between the inner wall and the outer wall; and
at least one internal element in the form of at least one partition element, the partition element dividing at least one cavity into two partial cavities; wherein the partition element is arranged only on the outer wall of the prosthesis socket.

19. The prosthesis socket of claim 18, wherein the at least one partition element has at least one opening that connects the two partial cavities.

20. The prosthesis socket of claim 19, wherein at least one support element extends through the at least one opening of the at least one partition element.
